(19) United States
(12) Patent Application Publication
(10) Pub. No.: US 2010/0210887 A1
(43) Pub. Date: Aug. 19, 2010

(54) CATALYST FOR PRODUCING A LIGHT OLEFIN AND METHOD FOR PRODUCING A LIGHT OLEFIN

(75) Inventors: Tomoko Shibata, Chiba (JP); Kenichi Wakui, Chiba (JP); Kinsho Furusawa, Chiba (JP); Tetsuya Saruwatari, Chiba (JP); Takashi Umeki, Chiba (JP); Masami Sawai, Chiba (JP); Nobuyuki Aoi, Tokyo (JP); Kazuo Osada, Tokyo (JP)

Correspondence Address: OBLON, SPIVAK, MCCLELLAND MAIER & NEUSTADT, L.L.P., 1940 DUKE STREET, ALEXANDRIA, VA, 22314, US

(73) Assignees: IDEMITSU KOSAN CO., LTD., Tokyo (JP); NIKKI-UNIVERSAL CO., LTD., Tokyo (JP)

(21) Appl. No.: 12/680,096

(22) PCT Filed: Oct. 16, 2008
(86) PCT No.: PCT/JP08/68717

§ 371 Date: Mar. 25, 2010

(30) Foreign Application Priority Data
Oct. 23, 2007 (JP) 2007-275409
Jun. 30, 2008 (JP) 2008-170214

(51) Int. Cl. C07C 1/20; B01J 29/40; C07C 1/00

(52) U.S. Cl. 585/640; 502/60; 585/500; 585/638

(57) ABSTRACT

A catalyst for producing a light olefin including a pentasil-type zeolite, alkaline earth metal atoms and aluminum atoms contained in the pentasil-type zeolite satisfying the following atomic ratio: [alkaline earth metal atom/aluminum atom]=0.2 to 15, the average value of the gradient of an adsorption isotherm of the pentasil-type zeolite measured by the nitrogen adsorption method being 30 or more at a relative pressure of 0.2 to 0.7.

### TECHNICAL FIELD

The invention relates to a catalyst for producing a light olefin and a method for producing a light olefin.

### BACKGROUND ART

A light olefin such as ethylene, propylene and butene is a crucially important compound as basic raw materials of various chemical products. As the method for producing a light olefin, a number of methods are reported in which oxygen-containing organic compounds such as methanol and dimethyl ether as raw materials are processed by using a catalyst.

In the above-mentioned method for producing a light olefin, zeolite has been used mainly as a catalyst. As for the zeolite to be used, a number of cases have been reported in which silicoaluminophosphate having a CHA structure (SAPO-34) and aluminosilicate having an MFI structure (ZSM-5) are used (Non-patent document 1 and Non-patent document 2).

Of these, SAPO-34 has a smaller pore size than ZSM-5. Therefore, carbonaceous substances are deposited on the surface thereof, and active sites (acid sites) which act effectively on the synthesis reaction of a light olefin are poisoned, whereby the life of the catalyst is shortened (coking deterioration). Therefore, in the method for producing a light olefin using SAPO-34 as a catalyst, a continuous regeneration method with a fluid bed reactor is employed, for example.

Meanwhile, as compared with SAPO-34, coking deterioration proceeds slowly in ZSM-5 having an MFI structure. Therefore, ZSM-5 can realize a method for producing a light olefin using a fixed bed reactor (Non-patent document 3). Since a fixed bed reactor has a simple structure as compared with a fluid bed reactor, it is economically advantageous in respect of construction costs or the like. For this reason, in the method for producing a light olefin using a zeolite having an MFI structure, studies have been made to further suppress coking.

Patent document 1 and Non-patent document 4 each disclose that coking deterioration can be suppressed by incorporating an alkaline earth metal such as calcium in a zeolite having an MFI structure. In addition, Patent document 1 and Non-patent document 5 each disclose that the catalyst life may be prolonged when the crystal size (average particle size) of a zeolite having an MFI structure, which is used in a catalyst, is small. In addition to the methods mentioned above, various studies have been made to suppress coking deterioration. However, a catalyst for producing a light olefin which has a satisfactorily long catalyst life has not yet been developed.

Patent document 1: JP-A-2005-138000

Non-patent document 1: Catalysis Today, vol. 106, 2005, p. 103, John Q. Chen et al.

Non-patent document 2: Microporous and Mesoporous Materials, vol. 29, 1999, p. 3, Michael Stocker

Non-patent document 3: Journal of the Japan Institute of Energy, vol. 84, 2005, p. 335, Makoto Inomata

Non-patent document 4: Summary of the 48^{th} annual meeting of the Japan Petroleum Institute, edited by the Japan Petroleum Institute, 2005, p. 96, Yusuke Watanabe, Koji Omata and Muneyoshi Yamada

Non-patent document 5: Report of the Japan Petroleum Institute, Vol. 34, 1991, p. 90, Kichinari Kawamura, Yasuo Kono, Kenji Matsuzaki, Tsuneji Sano and Haruo Takaya

An object of the invention is to provide a catalyst for producing a light olefin which is less likely to suffer from coking deterioration and has a long catalyst life.

Another object of the invention is to provide a method for producing a light olefin using as raw materials oxygen-containing organic compounds such as methanol and dimethyl ether.

### DISCLOSURE OF THE INVENTION

The inventors made extensive studies to solve the above-mentioned problems, and have found that, by using as a catalyst, a pentasil-type zeolite having specific physical properties, a light olefin can be produced stably for a long period of time using an oxygen-containing organic compound such as methanol and dimethyl ether as a raw material. The invention has been made based on this finding.

According to the invention, the following catalyst for producing a light olefin or the like can be provided.

1. A catalyst for producing a light olefin comprising a pentasil-type zeolite,

alkaline earth metal atoms and aluminum atoms contained in the pentasil-type zeolite satisfying the following atomic ratio:

[alkaline earth metal atom/aluminum atom]=0.2 to 15,

the average value of the gradient of an adsorption isotherm of the pentasil-type zeolite measured by the nitrogen adsorption method being 30 or more at a relative pressure of 0.2 to 0.7.

2. The catalyst for producing a light olefin according to 1, wherein the pentasil-type zeolite has an absorption maximum between 3650 cm⁻¹ and 3710 cm⁻¹ in an infrared absorption spectroscopic measurement by Fourier transform infrared spectroscopy.
3. The catalyst for producing a light olefin according to 1 or 2, the pentasil-type zeolite has an MFI structure.
4. The catalyst for producing a light olefin according to any one of 1 to 3, wherein silicon atoms and aluminum atoms contained in the pentasil-type zeolite satisfy the following atomic ratio:

[silicon atom/aluminum atom]=20 to 300.

5. The catalyst for producing a light olefin according to any one of 1 to 4, wherein the catalyst is obtained by a hydrothermal synthesis at a temperature of 150° C. or lower.
6. The catalyst for producing a light olefin according to any one of 1 to 5, wherein the catalyst is obtained by a hydrothermal synthesis using an organic silicon compound.
7. A method for producing a light olefin which uses the catalyst for producing a light olefin according to any one of 1 to 6.
8. The method for producing a light olefin according to 7, wherein a light olefin is produced by reacting an oxygen-containing organic compound having 1 to 4 carbon atoms and the catalyst for producing a light olefin.
9. The method for producing a light olefin according to 8, wherein the oxygen-containing organic compound having 1 to 4 carbon atoms comprises at least one of methanol, dimethyl ether and ethanol.
10. The method for producing a light olefin according to 8 or 9, wherein steam is supplied to the oxygen-containing hydrocarbon organic compound such that the weight ratio of the steam to the oxygen-containing organic compound satisfies the following equation:

[steam/oxygen-containing organic compound]=0.1 to 10.

The invention can provide a catalyst for producing a light olefin which is less likely to suffer from coking deterioration and has a long catalyst life.

The invention can provide a method for producing a light olefin using oxygen-containing organic compounds such as methanol and dimethyl ether.

### BRIEF DESCRIPTION OF THE DRAWINGS

FIG. 1 is a view showing adsorption isotherms of the zeolite prepared in Example 1 and Comparative Example 1 measured by the nitrogen adsorption method at a relative pressure of 0 to 1;

FIG. 2 is a view showing the results of infrared absorption spectroscopic measurement of the zeolite used in Examples 1 to 3 and Comparative Examples 1 to 4; and

FIG. 3 is a view showing the relationship between the average value of the gradient of the adsorption isotherm and the life of the catalyst [g-DME/g-catalyst] at a relative pressure of 0.2 to 0.7, as to the results obtained in Examples 1 and 3, and Comparative Examples 1 to 4.

### BEST MODE FOR CARRYING OUT THE INVENTION

The catalyst for producing a light olefin according to the invention is a catalyst comprising a pentasil-type zeolite, alkaline earth metal atoms and aluminum atoms contained in the pentasil-type zeolite satisfying the following atomic ratio:

[alkaline earth metal atom/aluminum atom]=0.2 to 15;

and the average value of the gradient the adsorption isotherm of the pentasil-type zeolite measured by the nitrogen adsorption method is 30 or more at a relative pressure of 0.2 to 0.7.

In the invention, the “relative pressure” is defined as [adsorption equilibrium pressure/saturated vapor pressure of nitrogen at 77K].

The pentasil zeolite is a zeolite composed of a combination of oxygen-containing five-membered rings. The pentasil-type zeolite of the invention contains an alkaline earth metal.

Examples of the alkaline earth metal contained in the pentasil-type zeolite of the invention include magnesium, calcium, strontium and barium, with calcium being preferable.

The alkaline earth metal and aluminum contained in the pentasil-type zeolite satisfy the following atomic ratio: [alkaline earth metal atom/aluminum atom]=0.2 to 15. The atomic ratio [alkaline earth metal atom/aluminum atom] is preferably in the range of 0.3 to 10, more preferably in the range of 0.5 to 5.

If the atomic ratio [alkaline earth metal atom/aluminum atom] is less than 0.2, the life of a catalyst is shortened, and the yield of a light olefin may be decreased. If the atomic ratio [alkaline earth metal atom/aluminum atom] exceeds 15, preparation of a catalyst, which will be mentioned later, may be difficult.

The average value of the adsorption isotherm of a pentasil-type zeolite measured by the nitrogen adsorption method is 30 or more at a relative pressure of 0.2 to 0.7.

The nitrogen adsorption method is a method for measuring the specific area of powder particles or the like.

In the invention, it is possible to use the nitrogen adsorption method which is generally conducted. For example, the nitrogen adsorption method described in “Science and Application of Adsorption (2003, page 60, Kodansha Scientific Ltd., Yoshio Ono, Isao Suzuki) may be used. An adsorption isotherm can be obtained by taking the adsorbed amount of nitrogen measured by the nitrogen adsorption method and the relative pressure at the measurement temperature (normally, 77K) on the ordinate and the abscissa, respectively. This measurement can be performed by means of a commercially available adsorption measurement apparatus manufactured by BEL Japan, Inc., Yuasa Ionics, Inc., or other manufacturers.

There are no specific restrictions on the average value of the gradient of an adsorption isotherm of the pentasil-type zeolite measured by the nitrogen adsorption method insofar as it is 30 or more at a relative pressure of 0.2 to 0.7. However, the upper limit thereof is 300, for example.

As for the measurement results obtained by the above-mentioned nitrogen adsorption method, an adsorption isotherm can be obtained by taking a relative pressure on the abscissa and by taking an adsorbed nitrogen amount on the ordinate. Normally, an adsorption isotherm is obtained by gradually increasing the relative pressure from a low pressure to a high pressure.

In the invention, the average value of the gradient of above-mentioned adsorption isotherm at a relative pressure of 0.2 to 0.7 is a value calculated by the following formula (I):

$$Average value of the gradient of adsorption isotherma = \frac{Vp ( 0.7 ) - Vp ( 0.2 )}{0.7 - 0.2} \qquad ( 1 )$$

wherein Vp (0.7) is the adsorbed nitrogen amount [cm³] per gram of zeolite at a relative pressure of 0.7, and Vp (0.2) is the adsorbed nitrogen amount [cm³] per gram of zeolite at a relative pressure of 0.2.

In the above-mentioned formula (1), the adsorbed nitrogen amount [cm³] shown by Vp (0.7) and Vp (0.2) are nitrogen volumes obtained after a 0° C. and 1 atmospheric pressure conversion, and can be obtained directly from the adsorption isotherm.

In the invention, the average value of the gradient of the above-mentioned adsorption isotherm at a relative pressure of 0.2 to 0.7 is 30 or more, more preferably 40 or more. If the average value of the gradient of the adsorption isotherm is less than 30, effects of suppressing coking deterioration cannot be obtained sufficiently.

The pentasil-type zeolite has an absorption maximum between 3650 cm⁻¹ and 3710 cm⁻¹ in an infrared absorption spectroscopic measurement analysis by Fourier transform infrared spectroscopy. In this region, the stretching vibration of the hydroxyl group of the zeolite is observed. The absorption maximum in the above-mentioned region is assumed to be a new acid site formed by the acid site based on an alkaline earth metal and silicon and aluminum in the zeolite.

Meanwhile, the infrared absorption spectroscopic measurement by Fourier transform infrared spectroscopy can be performed by a method described in “Trends in Physical Chemistry, vol. 1, page 133, 1990, T. Sano, H. Okado, and H. Takaya”. For example, it can be performed, by a commercially available apparatus manufactured by JASCO Corporation or other manufacturers.

The zeolite used in the invention is a pentasil-type zeolite. Examples thereof include a zeolite having an MFI structure such as ZSM-5 and ZSM-11 (“Science and Application of Zeolite”, 1987, page 87, Kodansha Scientific, Hiroo Tominaga).

Meanwhile, the MFI structure is the name of a zeolite skeleton structure defined by the International Zeolite Association.

It is preferred that the silicon and aluminum contained in a pentasil-type zeolite satisfy the atomic ratio [silicon atom/aluminum atom]=20 to 300.

If the atomic ratio [silicon atom/aluminum atom] is less than 20, deposition of a carbonaceous product on a catalyst is promoted due to an increase in effective acid sites. As a result, catalyst life may be shortened at an early stage. On the other hand, if the atomic ratio [silicon atom/aluminum atom] exceeds 300, catalytic activity may be deteriorated due to a decrease in effective acid sites.

The pentasil-type zeolite contained in the catalyst for producing a light olefin of the invention can be formed, for example, by the hydrothermal synthesis method. The hydrothermal synthesis method is a method for synthesizing a compound in the presence of heated water, and is widely used for the synthesis of zeolites.

Specifically, silica sources, aluminum sources, water, alkaline salts, alkaline earth metal salts, a structure-defining agent (template) or the like are put in an autoclave, and heated and stirred at a temperature of around 60° C. to 200° C. for 1 to 200 hours under self-pressure conditions, thereby to proceed hydrothermal synthesis. The reaction product obtained by the hydrothermal synthesis is separated by filtration or centrifugation, washed with water, dried and baked at 300 to 700° C. for 1 to 100 hours, whereby the zeolite of the invention is prepared.

Meanwhile, the above-mentioned zeolite may further be subjected to an acid treatment or ion-exchanged to allow it to be an ammonium type-zeolite, and the resultant is dried and baked again. For the acid treatment, an inorganic acid such as hydrochloric acid, sulfuric acid and nitric acid or an organic acid such as formic acid and acetic acid are used. Of these, hydrochloric acid is preferable. The ion exchange to make the zeolite an ammonium-type zeolite is performed in ammonia water, an aqueous solution of an ammonium salt such as ammonium chloride, ammonium nitrate and ammonium sulfate. Due to the addition of the above-mentioned baking step, the zeolite can be of proton type.

As the silica source, in addition to colloidal silica and water glass, an organic silicon compound or the like can be given. An organic silicon compound may preferably be used. Specific examples of the organic silicon compounds include alkoxide compounds such as tetraethoxysilane ((C₂H₅O)₄Si) and tetramethoxysilane ((CH₃O)₄Si).

Examples of the aluminum source include alumina sol, boehmite and organic aluminum compounds.

As the structure-defining agent, various quaternary ammonium salts (for example, tetrapropylammonium bromide, tetrapropylammonium hydroxide), amines (triethylamine) or the like can be given. The synthesis can be performed without using the structure-defining agent.

Examples of the alkaline salts include sodium hydroxide and potassium hydroxide. Examples of the alkaline earth metal salts include nitric acid salts and acetic acid salts of an alkaline earth metal.

The above-mentioned silica source, aluminum source, structure-defining agent, alkaline salts and alkaline earth metal salts may be used either singly or in combination of two or more.

In the synthesis of the pentasil-type zeolite, seed crystals of zeolite may be added to improve crystallinity, as well as to shorten the synthesis time. Although MFI seed crystals are suitable as the seed crystals, seed crystals with other structures, such as FAU seed crystals and MOR seed crystals, may be used (FAU structure and MOR structure are the name of the structure of a skeleton defined by the International Zeolite Association). The average particle size of the seed crystal is preferably 1.5 μm or less, with 0.5 μm or less being more preferable.

It is preferred that the amount ratio in the synthesis of the pentasil-type zeolite be set such that the following atomic ratio and molar ratio are satisfied. Atomic ratio [silicon/aluminum]=20 to 300, atomic ratio [alkali metal atom/aluminum atom]>1, molar ratio [structure-defining agent/aluminum]>1 and molar ratio [water/(alkali metal+structure-defining agent)]=2 to 30.

In the invention, it is preferred that the pentasil-type zeolite be synthesized by a method in which an organic silicon compound is used as the silica source, a raw material mixture is sufficiently aged before heating in an autoclave, and the mixture is hydrothermally synthesized at a further lower heating temperature. Here, the “aging” means a procedure in which a raw material mixture is continuously stirred while keeping it at around room temperature. It is preferred that aging be performed for 2 hours or longer and heating temperature at the time of the hydrothermal synthesis be 150° C. or lower. The conditions are not limited thereto, and various conditions may be appropriately selected according to the raw materials used.

By using the catalyst for producing a light olefin of the invention, a light olefin such as ethylene and propylene can be produced.

Production of the above-mentioned light olefin is performed by a method in which a fixed bed, moving bed or fluid bed type reactor is used as a reactor, and raw material hydrocarbons are supplied to a catalyst layer which has been filled with the catalyst of the invention.

It is preferred that an oxygen-containing organic compound having 1 to 4 carbon atoms be used as a raw material. An oxygen-containing organic compound comprising at least one of methanol, ethanol and dimethyl ether is more preferable, with an oxygen-containing organic compound consisting essentially of at least one of methanol, ethanol and dimethyl ether being most preferable.

In addition, in the catalyst of the invention, it is preferred that steam be supplied such that the weight ratio of the steam to the above-mentioned oxygen-containing organic compound satisfies the equation of [steam/oxygen-containing organic compound]=0.1 to 10. The supplied material is not limited to steam, and nitrogen, hydrogen, helium or the like may be supplied if the need arises.

The reaction temperature of the catalyst for producing a light olefin of the invention and the hydrocarbons as the raw material is normally 300 to 750° C., preferably 400 to 650° C., more preferably 450 to 600° C.

By practicing the method for producing a light olefin of the invention under the above-mentioned conditions, it is possible to allow the catalyst of the invention to be less likely to suffer from coking deterioration and have a long catalyst life.

### EXAMPLES

### Example 1

### Synthesis of Zeolite

0.25 g of an aqueous solution of sodium hydroxide (concentration: 10 wt %) and 1.2 g of de-ionized water were put in a Teflon (registered trade mark)-made container, followed by stirring to obtain a homogenous aqueous solution. To this aqueous solution, 50 g of an aqueous solution of tetrapropylammonium hydroxide (concentration: 10 wt %) was added. To the resultant, 0.06 g of aluminum hydroxide and 0.458 g of calcium nitrate tetrahydrate were added and stirred. When the solution became homogeneous, 16.36 g of tetraethoxysilane was added and stirred for 1 hour. After the lapse of 1 hour, gel in the container, which was composed of the added raw materials, was put in an autoclave (equipped with a Teflon-made inner tube, internal volume: 100 mL). Then, the autoclave was installed in a heating chamber of a hydrothermal synthesis apparatus (manufactured by HIRO Company). While rotating the entire autoclave at 30 rpm, heating and stirring were conducted at room temperature (25° C.) for 24 hours and further at 100° C. for 48 hours. After the completion of heating and stirring, the autoclave was allowed to cool, and the contents were collected.

The contents (white suspension) thus collected were placed in a 100 mL-eggplant flask, and the water was evaporated and distilled off by means of an evaporator, and a white solid product was collected. The collected white solid product was dried at 120° C. overnight. Then, the white solid product was air-baked at 550° C. for 6 hours in a muffle furnace, whereby white powder was obtained. The powder was ion-exchanged at 80° C. for 6 hours using an aqueous 0.5M ammonium nitrate solution, and then baked (550° C. for 6 hours), whereby ZAC-1, which was proton-type zeolite powder, was obtained.

### [Evaluation of Zeolite]

The thus obtained ZAC-1 was subjected to X-ray diffraction analysis. Measurement was conducted by means of an X-ray diffraction apparatus (RINT-Ultima III, manufactured by Rigaku Corporation). The results confirmed that ZAC-1 was an MEI zeolite.

The conditions of the X-ray diffraction analysis were as follows.

X rays: Cu-Kα rays (monochromated by means of a graphite monochrometer)

Wavelength: λ=1.540 Å, output: 40 kV, 40 mA

Scanning: scanning step interval 0.02°

Scanning speed: 1 sec/step

Measurement range: 5 to 80°

The composition of the resulting ZAC-1 was analyzed by the ICP emission spectroscopic method. As a result of the measurement by means of an ICP emission spectroscopy apparatus (SPS 5100, manufactured by SII Nanotechnology, Inc), it was confirmed that ZAC-1 contained calcium, and the atomic ratio [calcium atom/aluminum atom] was 3.1, and the atomic ratio [silicon atom/aluminum atom] was 138.

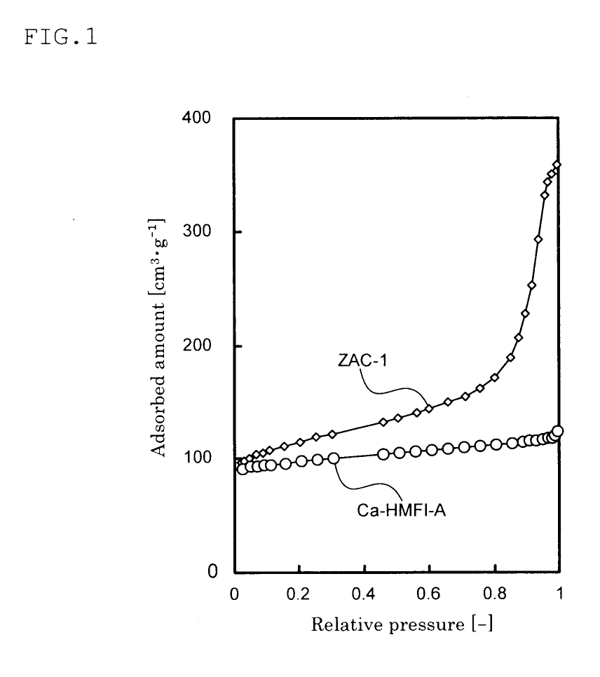

For the resulting ZAC-1, the adsorbed nitrogen amount was measured by the nitrogen adsorption method, whereby an adsorption isotherm was obtained. By using Autosorb-6 (manufactured by Yuasa Ionics, Inc.) and according to a method described in “Catalyst, Vol. 26, No. 6, page 495, (Reference catalyst committee of Catalyst Society of Japan, 1984)”, the adsorbed nitrogen amount was measured at a liquid nitrogen temperature (77K) and at a nitrogen pressure of 1 kPa to 100 kPa. The resulting adsorption isotherm is shown in FIG. 1. From FIG. 1, the gradient of the adsorption isotherm of ZAC-1 was confirmed to be 79 at a relative pressure of 0.2 to 0.7.

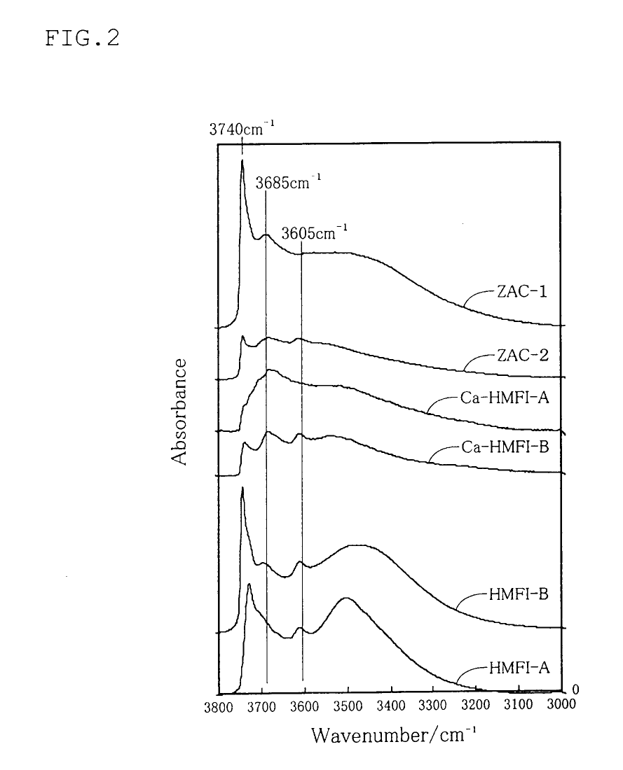

For the resulting ZAC-1, an infrared absorption spectroscopic measurement by Fourier transform infrared spectroscopy was conducted. ZAC-1 was shaped into a disc-like form, and placed in an evacuatable infrared absorption measurement cell. Evacuation baking was conducted at 400° C. for 2 hours. After baking and cooling, by means of a Fourier transform infrared spectrophotometer (Model: FT/IR-550, manufactured by Jasco Corporation), an infrared absorption measurement was conducted 200 times (an integrated number of times) for a wavenumber range of 3000 cm⁻¹ to 4000 cm⁻¹ at room temperature and at a scanning speed of 4 mm/sec. The results are shown in FIG. 2.

For comparison, an infrared absorption spectroscopic measurement was similarly conducted for a commercially available proton-type MFI zeolite, which will be used in Comparative Example 3 given later (HMFI-A, manufactured by Nikki Universal Co., Ltd., Si/Al molar ratio=175). The results are shown in FIG. 2.

In the case of the commercially available HMFI-A, a peak derived from an acidic hydroxyl group was observed at around 3605 cm⁻¹ and a peak derived from a silanol group was observed at around 3740 cm⁻¹. On the other hand, in the case of ZAC-1, whereas the peak derived from a silanol group at around 3740 cm⁻¹ was observed, the peak derived from an acidic hydroxyl group at around 3605 cm⁻¹ was not observed, and another peak was observed at around 3685 cm⁻¹. That is, the state of the active site of ZAC-1 was confirmed to be different from that of the normal HMFI zeolite.

### [Production of a Light Olefin]

The ZAC-1 zeolite powder was solidified by compression at a load of 60 MPa, and the resultant was ground in a mortor, and classified into particles with a diameter of about 1 mm. 1 g of the resulting granulated molded particle was put in a stainless-made reactor having an inner diameter of 14 mm (equipped with an interpolated tube having an outer diameter of 3 mm for accommodating a thermocouple), whereby a catalyst layer with a thickness of about 15 mm was obtained. In order to retain the catalyst layer, the upper and lower parts of the catalyst layer were filled with quartz wool. Other parts of the reactor were filled with alumina ball having a diameter of 2 mm (Model A-901, manufactured by Fujimi Inc.). While flowing nitrogen to this reactor at a rate of 60 cm³/min (after conversion at 0° C. and 1 atmospheric pressure, the same was applied hereinbelow), and the temperature of the catalyst layer was elevated to 600° C. and baking was performed at this temperature for 1 hour. After baking, while keeping the temperature of the catalyst layer at 450° C., dimethyl ether as a raw material was supplied at a flow rate of 48 cm³/min, and further, nitrogen was supplied at a flow rate of 48 cm³/min to allow the dimethyl ether to react.

As for the analysis of the reaction products, after the lapse of a predetermined time from the start of circulation of the raw materials, the gas at the reactor outlet was subjected to on-line sampling (sampling was performed after complete gasification of the generated products). The yield of the generated products and the conversion ratio of the raw material were analyzed by gas chromatography.

Meanwhile, in the invention, the yield of the generated products and the conversion ratio of the raw material are defined by the following formula:

Yield of generated products (carbon %)=(molar amount of carbon in the generated light olefin/molar amount of carbon in the supplied raw material)×100

Raw material conversion ratio (%)=(1−weight of unreacted raw material/weight of supplied raw material)×100 (methanol formed in a small amount was calculated as the raw material (converted to dimethyl ether).)

The conversion ratio of dimethyl ether at the initial stage of the reaction was generally stable at 95% or more (maximum: 100%). However, due to the reaction for a long period of time, coking deterioration of the catalyst proceeded, and at a certain point, the conversion ratio became less than 95%, and thereafter, the activity lowered rapidly.

In the invention, the amount of dimethyl ether (DME) which was reacted per gram of the catalyst (zeolite powder) during a period of time from the start of flowing dimethyl ether on the catalyst until the conversion ratio of dimethyl ether was lowered to less than 95% was defined as the catalyst life [unit: g-DME/g-catalyst].

The composition of the gas at the reactor outlet was analyzed 1.5 hours after the start of the reaction. The results showed that the conversion ratio of dimethyl ether was 100% and the yield of the generated products ((ethylene+propylene+butene)/dimethyl ether)) was 67.2%. While keeping the temperature of the catalyst layer at 450° C., the reaction was continued, and the composition of the generated gas at the reactor outlet was occasionally analyzed. In the composition analysis of the generated gas at the outlet of the rector, the total amount of dimethyl ether reacted until the conversion ratio of dimethyl ether was lowered to less than 95% was measured. As a result, it was found that the catalyst life was 1539 [g-DME/g-catalyst]. The results are shown in Table 1.

Meanwhile, in Table 1, as for the catalysts used for producing a light olefin, the case where an absorption maximum was observed between 3650 cm⁻¹ and 3710 cm⁻¹ was indicated as “observed”, and the case where an absorption maximum between 3650 cm⁻¹ and 3710 cm⁻¹ was not observed was indicated as “not observed”. Similarly, the case where dilution by steam was conducted was indicated as “performed” and the case where dilution with steam was not conducted was indicated as “not performed”.

### Comparative Example 1

### Synthesis of Zeolite

According to the method described in the “Journal of the Chemical Society of Japan, Vol. 1, page 25, 1987”, colloidal silica (Cataloid SI-350, manufactured by Catalysts & Chemicals Industries Co., Ltd.), aluminum nitrate nonahydrate, calcium nitrate tetrahydrate, sodium hydroxide and tetrapropylammonium bromide (TPABr) were mixed to prepare a slurry having the following molar composition:

Si/Al=100,OH—/SiO₂=0.1,TPABr/SiO₂=0.1,H₂O/SiO₂=40,Ca/Si=0.025

The resulting slurry was placed in a 2 L-autoclave, and heated at 160° C. for 16 hours with stirring to perform a hydrothermal synthesis. The resulting products were thoroughly washed with ion exchange water, dried at 110° C., and baked at 600° C. for 4 hours. The collected powder was ion-exchanged at 80° C. for 6 hours using a 0.5 M aqueous ammonium nitrate solution, followed by baking (550° C. for 6 hours), whereby Ca-HMFI-A, proton-type zeolite powder, was obtained.

### [Evaluation of Zeolite]

The resulting Ca-HMFI-A was evaluated in the same manner as in Example 1.

As a result, it was confirmed that Ca-HMFI-A was MFI zeolite, and the atomic ratio [calcium atom/aluminum atom] was 1.7 and the atomic ratio [silicon atom/aluminum atom] was 91. The adsorption isotherm and the results of the infrared absorption spectroscopic measurement of Ca-HMFI-A are shown in FIG. 1 and FIG. 2, respectively.

From FIG. 2, it was confirmed that Ca-HMFI-A had an absorption peak at around 3685 cm⁻¹ as in the case of ZAC-1. However, from FIG. 1, the gradient of the adsorption isotherm at a relative pressure of 0.2 to 0.7 was confirmed to be as small as 24.

### [Production of a Light Olefin]

A light olefin was prepared in the same manner as in Example 1, except that Ca-HMFI-A was used instead of ZAC-1.

1.5 hours after the start of the reaction, the composition of the gas at the reactor outlet was analyzed. It was found that the conversion ratio of dimethyl ether was 99.5% and the yield of the generated products ((ethylene+propylene+butene)/dimethyl ether) was 55.6%. The total amount of dimethyl ether reacted until the conversion ratio of dimethyl ether was lowered to less than 95% was measured. As a result, it was found that the catalyst life was 987 [g-DME/g-catalyst]. The results are shown in Table 1.

From Example 1 and Comparative Example 1, it was found that, even though the zeolite contained an alkaline earth metal atom, the catalyst life was short if the gradient of the adsorption isotherm as specified in the invention was small.

### Comparative Example 2

### Synthesis of Zeolite

According to the method described in Example 1 of JP-A-2005-138000, a zeolite raw material liquid composed of 9.50 g of Al(NO₃)₃.9H₂O and 10.92 g of Ca(CH₃COO)₂.H₂O was dissolved in 750 g of water to prepare an aqueous solution of the zeolite raw material. To this zeolite raw material solution, a solution obtained by dissolving 500 g of Cataloid Si-30 water glass (manufactured by Catalysts & Chemicals Industries Co., Ltd) in 333 g of water, 177.5 g of 6 mass % of an aqueous NaOH solution, 317.6 g of 21.3 mass % of an aqueous tetrapropylammonium bromide solution, and as the zeolite seed crystal, 15.0 g of ammonium-type zeolite having an MFI structure having an average particle size of 0.5 μm (manufactured by Zeolyst International, Si/Al atomic ratio: 70) (an amount corresponding to 10 mass % of the amount of the zeolite catalyst which was synthesized without adding seed crystals) were added with stirring, whereby an aqueous gel mixture was obtained.

The resulting aqueous gel mixture was placed in a 3 L-autoclave, and stirred at 160° C. at a self pressure for 18 hours to perform hydrothermal synthesis. A white solid product obtained by the hydrothermal synthesis was filtered and washed with water, dried at 120° C. for 5 hours, and baked in air at 520° C. for 10 hours. The resulting baked product was immersed in 0.6N hydrochloric acid, and stirred at room temperature for 24 hours to prepare a proton-type zeolite. Thereafter, the product was filtered and washed with water, dried at 120° C. for 5 hours, and baked at 520° C. for 10 hours in air, whereby Ca-HMFI-B, proton-type zeolite powder, was obtained.

### [Evaluation of Zeolite]

The resulting Ca-HMFI-B was evaluated in the same manner as in Example 1.

As a result, it was confirmed that Ca-HMFI-B was an MFI zeolite having an atomic ratio [calcium atom/aluminum atom] of 0.9 and an atomic ratio [silicon atom/aluminum atom] of 73. The results of the infrared absorption spectrometric measurement of Ca-HMFI-B are shown in FIG. 2.

From FIG. 2, it was confirmed that Ca-HMFI-B had an absorption peak at around 3685 cm⁻¹ as in the case of ZAC-1. Unlike ZAC-1, however, Ca-HMFI-B had another absorption peak at around 3605 cm⁻¹. In addition, the gradient of the adsorption isotherm at a relative pressure of 0.2 to 0.7 was as small as 28.

### [Production of a Light Olefin]

A light olefin was prepared in the same manner as in Example 1, except that Ca-HMFI-B was used instead of ZAC-1.

1.5 hours after the start of the reaction, the composition of the gas at the reactor outlet was analyzed. It was found that the conversion ratio of dimethyl ether was 99.8% and the yield of the generated products ((ethylene+propylene+butene)/dimethyl ether) was 58.3%. The total amount of dimethyl ether reacted until the conversion ratio of dimethyl ether was lowered to less than 95% was measured. As a result, it was found that the catalyst life was 964 [g-DME/g-catalyst]. The results are shown in Table 1.

From Example 1 and Comparative Example 2, even if the zeolite was reduced in size, the zeolite had a short life if the gradient of the adsorption isotherm as specified in the invention was small.

### Comparative Example 3

### Production of a Light Olefin

A light olefin was prepared in the same manner as in Example 1, except that HMFI-A (manufactured by Nikki Universal Co., Ltd., atomic ratio [Si atom/aluminum atom=175], the gradient of the adsorption isotherm at a relative pressure of 0.2 to 0.7 was 28), which was a commercially available proton-type zeolite containing no alkaline earth metal, was used instead of ZAC-1.

1.5 hours after the start of the reaction, the composition of the gas at the reactor outlet was analyzed. It was found that the conversion ratio of dimethyl ether was 100% and the yield of the generated products ((ethylene+propylene+butene)/dimethyl ether) was 57.3%. The total amount of dimethyl ether reacted until the conversion ratio of dimethyl ether was lowered to less than 95% or less was measured. As a result, it was found that the catalyst life was 139 [g-DME/g-catalyst]. The results are shown in Table 1.

From Example 1 and Comparative Example 3, the catalyst which contained no alkaline earth metal and had a small gradient of the adsorption isotherm had a short life.

### Comparative Example 4

### Synthesis of Zeolite

HMFI-B, proton-type zeolite powder containing no alkaline earth metal, was obtained in the same manner as in Example 1, except that calcium nitrate tetrahydrate was not added.

### [Evaluation of Zeolite]

The resulting HMFI-B was evaluated in the same manner as in Example 1.

As a result, it was confirmed that HMFI-B was an MFI zeolite having an atomic ratio [silicon atom/aluminum atom] of 118. The results of the infrared absorption spectrometric measurement of HMFI-B are shown in FIG. 2.

From FIG. 2, it was confirmed that HMFI-B had a peak derived from an acidic OH at around 3605 cm⁻¹ and a peak derived from silanol at around 3740 cm⁻¹. An absorption peak was observed also at around 3685 cm⁻¹. Since HMFI-B contained no alkaline earth metal, it is assumed that the peak at around 3685 cm¹ was not a peak derived from an alkaline earth metal, but it is a peak derived from silanol which was different from the silanol of which the peak derived therefrom was observed at around 3740 cm⁻¹. In addition, the gradient of the adsorption isotherm at a relative pressure of 0.2 to 0.7 was confirmed to be as large as 62.

### [Production of a Light Olefin]

A light olefin was prepared in the same manner as in Example 1, except that HMFI-B was used instead of ZAC-1.

1.5 hours after the start of the reaction, the composition of the gas at the reactor outlet was analyzed. It was found that the conversion ratio of dimethyl ether was 100% and the yield of the generated products ((ethylene+propylene+butene)/dimethyl ether) was 49.3%. The total amount of dimethyl ether reacted until the conversion ratio of dimethyl ether was lowered to less than 95% was measured. As a result, it was found that the catalyst life was 639 [g-DME/g-catalyst]. The results are shown in Table 1.

From Example 1 and Comparative Example 4, it was found that, in the case of a catalyst which contained no alkaline earth metal, even if it was composed of a zeolite having a large gradient of the adsorption isotherm at a relative pressure of 0.2 to 0.7 and had an absorption maximum (peak) between 3650 cm⁻¹ and 3710 cm⁻¹, the life thereof was short.

### Example 2

### Production of a Light Olefin

In the same manner as in Example 1, a reactor was filled with ZAC-1 zeolite powder prepared in Example 1. While flowing nitrogen to this reactor at a rate of 60 cm³/min (after conversion at 0° C. and 1 atmosphere, the same was applied hereinbelow), the temperature of the catalyst layer was elevated to 600° C. and baking was performed at this temperature for 1 hour. After baking, while keeping the temperature of the catalyst layer at 450° C., dimethyl ether as a raw material was supplied at a flow rate of 24 cm³/min, nitrogen was supplied at a flow rate of 9.6 cm³/min, and steam was supplied at a flow rate of 62.2 cm³/min, whereby dimethyl ether was allowed to react.

As for the above-mentioned steam, the steam was prepared by passing de-ionized water through a gasification apparatus at a flow rate of 3 mL/hour and the resulting steam was supplied to the reactor.

1.5 hours after the start of the reaction, the composition of the gas at the reactor outlet was analyzed. It was found that the conversion ratio of dimethyl ether was 96.9% and the yield of the generated products ((ethylene+propylene+butene)/dimethyl ether) was 55.9%. The reaction was continued while keeping the temperature of the catalyst layer at 450° C., and the composition of the gas at the outlet of the reactor was occasionally analyzed. As for the composition of the gas, when the total amount of reacted dimethyl ether became 3000 [g-DME/g-catalyst], the conversion ratio of dimethyl ether was 96.7% and the yield of the generated products was 54.7%. No deterioration was observed in zeolite. The temperature of the catalyst layer was elevated to 530° C. and supply of the steam was stopped. While supplying dimethyl ether at a flow rate of 48 cm³/min and supplying nitrogen at a flow rate of 48 cm³/min, the reaction of dimethyl ether was further continued. 1.5 hours after the temperature elevation, composition of the gas at the reactor outlet was analyzed. As a result, it was found that the conversion ratio of dimethyl ether was 100% and the yield of the generated products ((ethylene+propylene+butene)/dimethyl ether) was 71.1%. As for the composition of the gas at the outlet of the reactor when the total reacted amount of dimethyl ether became 4200 [g-DME/g-catalyst], the conversion ratio of dimethyl ether was 100% and the yield of generated products was 67.6%, and no deterioration in zeolite was observed. The results are shown in Table 1.

The zeolite of the invention hardly deteriorated even if steam was introduced and the reaction temperature was high.

### Example 3

### Synthesis of Zeolite

13.97 g of an aqueous solution of tetrapropylammonium hydroxide (concentration: 14.5 wt %) and 2.66 g of tetrapropylammonium bromide were put in a Teflon (registered trade mark)-made container, followed by stirring to obtain a homogenous aqueous solution. To this aqueous solution, 0.049 g of aluminum hydroxide and 0.355 g of calcium nitrate tetrahydrate were added and stirred. When the solution became homogeneous, 0.038 g of an aqueous solution of sodium hydroxide (concentration: 50 wt %) and 10 g of colloidal silica (Ludox AS-40, manufactured by Sigma-Aldrich Corp.) were added and stirred for two hours. After the lapse of 1 hour, gel in the container was placed in an autoclave (equipped with a Teflon-made inner tube, internal volume: 100 mL). Then, the autoclave was installed in a heating chamber of a hydrothermal synthesis apparatus (manufactured by HIRO Company). While rotating the entire autoclave at 20 rpm, the temperature was elevated to 120° C. over 60 hours, and retained at 120° C. for 6 hours. After the completion of heating and stirring, the autoclave was allowed to cool, and a white solid product was collected by centrifugation at 2000 rpm for 30 minutes.

The collected white solid product was dried at 120° C. overnight. Then, the white solid product was air-baked at 550° C. for 6 hours in a muffle furnace, whereby white powder was obtained. The powder was ion-exchanged at 80° C. for 7 hours using a 0.5M aqueous ammonium nitrate solution, followed by baking (550° C. for 6 hours), whereby ZAC-2, proton-type zeolite powder, was obtained.

### [Evaluation of Zeolite]

The resulting ZAC-2 was evaluated in the same manner as in Example 1.

As a result, it was confirmed that ZAC-2 was an MFI zeolite and had an atomic ratio [calcium atom/aluminum atom] of 0.34 and an atomic ratio [silicon atom/aluminum atom] of 110. The results of the infrared absorption spectroscopic measurement of ZAC-2 are shown in FIG. 2.

From FIG. 2, it was confirmed that, as in the case of ZAC-1, ZAC-2 had an absorption maximum at around 3685 cm⁻¹ and the gradient of the adsorption isotherm at a relative pressure of 0.2 to 0.7 was as large as 46.

### [Production of a Light Olefin]

A light olefin was produced in the same manner as in Example 1, except that ZAC-2 was used instead of ZAC-1.

1.5 hours after the start of the reaction, the composition of the gas at the reactor outlet was analyzed. It was found that the conversion ratio of dimethyl ether was 100% and the yield of the generated products ((ethylene+propylene+butene)/dimethyl ether) was 56.6%. The total amount of dimethyl ether reacted until the conversion ratio of dimethyl ether was lowered to less than 95% was measured. As a result, it was found that the catalyst life was 1551 [g-DME/g-catalyst]. The results are shown in Table 1.

### Example 4

### Production of a Light Olefin

In the same manner as in Example 1, a reactor was filled with ZAC-1 zeolite powder prepared in Example 1. While flowing nitrogen to this reactor at a rate of 60 cm³/min (after conversion at 0° C. and 1 atmosphere, the same was applied hereinbelow), and the temperature of the catalyst layer was elevated to 600° C. and baking was performed at this temperature for 1 hour. After baking, while keeping the temperature of the catalyst layer at 500° C., ethanol as a raw material was supplied at a flow rate of 16.6 cm³/min, nitrogen was supplied at a flow rate of 20 cm³/min, and steam was supplied at a flow rate of 42.5 cm³/min, whereby the ethanol was allowed to react.

As for the above-mentioned ethanol, a 50 wt % aqueous solution of ethanol was supplied to the reactor at a flow rate of 4.1 g/hour by means of micropump.

1 hour after the start of the reaction, the composition of the gas at the reactor outlet was analyzed. It was found that the conversion ratio of ethanol was 100% and the yield of the generated products ((ethylene+propylene+butene)/ethanol) was 99.9%. The reaction was continued while keeping the temperature of the catalyst layer at 500° C., the composition of the gas at the reactor outlet was occasionally analyzed. The conversion ratio of ethanol at a point when the amount of reacted ethanol per gram of catalyst became 1046 g (510 hours after the start of the reaction) was 100%. The yield of the generated products ((ethylene+propylene+butene)/ethanol) was 99.8%. From the above results, it was confirmed that the zeolite of the invention suffered from only a small degree of deterioration even when ethanol was used as a raw material.

TABLE 1

| | Example 1 | Com. Ex. 1 | Com. Ex. 2 | Com. Ex. 3 | Com. Ex. 4 | Example 2 | Example 3 |
|---|---|---|---|---|---|---|---|
| Catalyst | ZAC-1 | Ca-HMFI-A | Ca-HMFI-B | HMFI-A | HMFI-B | ZAC-1 | ZAC-2 |
| Atomic ratio | 3.1 | 1.7 | 0.9 | — | — | 3.1 | 0.34 |
| [Ca/Al] | | | | | | | |
| Atomic ratio | 138 | 91 | 73 | 175 | 118 | 138 | 110 |
| [Si/Al] | | | | | | | |
| Gradient of an | 79 | 24 | 28 | 28 | 62 | 79 | 46 |
| adsorption | | | | | | | |
| isotherm | | | | | | | |
| Absorption maximum | Observed | Observed | Observed | Not | Observed | Observed | Observed |
| between 3650 cm⁻¹ | | | | Observed | | | |
| and 3710 cm⁻¹ | | | | | | | |
| Dilution with steam | Not | Not | Not | Not | Not | Performed | Not |
| | performed | performed | performed | performed | performed | | performed |
| Catalyst life | 1539 | 987 | 964 | 139 | 639 | >4200 | 1551 |
| [g-DME/g-catalyst] | | | | | | | |

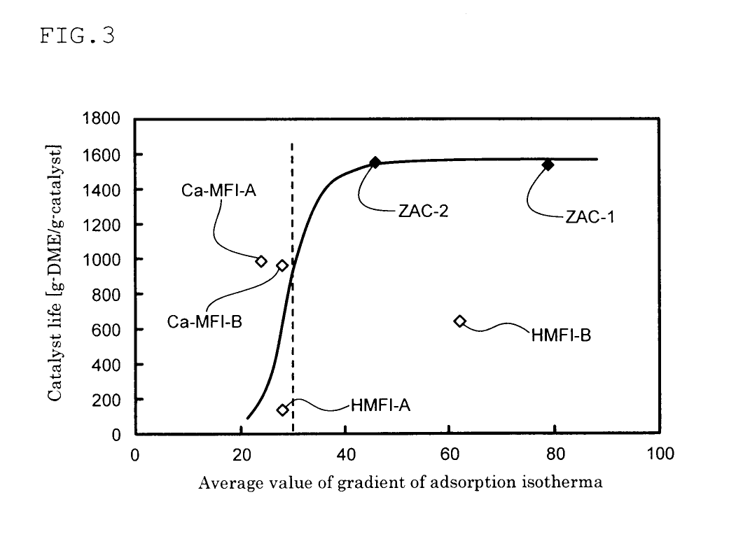

FIG. 3 shows, for the results obtained in Examples 1 and 3, and Comparative Examples 1 to 4, the relationship between the average of the gradient of an adsorption isotherm at a relative pressure of 0.2 to 0.7 and the catalyst life [g-DME/g-catalyst]. From FIG. 3, it was confirmed that the catalyst life significantly lowered from a point at which the average of the gradient of an adsorption isotherm was around 30.

### INDUSTRIAL APPLICABILITY

The catalyst for producing a light olefin of the invention can use as a raw material an oxygen-containing organic compound, and is capable of subjecting the oxygen-containing organic compound to contact decomposition, thereby producing a light olefin in a high yield. Furthermore, due to the long catalyst life thereof, the regeneration cycle of the catalyst for producing a light olefin of the invention is prolonged and the number of regeneration is reduced, whereby production efficiency can be improved and production cost can be reduced.

1. A catalyst for producing a light olefin comprising a pentasil-type zeolite,
wherein the alkaline earth metal atoms and aluminum atoms contained in the pentasil-type zeolite satisfy the following atomic ratio:
[alkaline earth metal atom/aluminum atom]=0.2 to 15,
and the average value of the gradient of an adsorption isotherm of the pentasil-type zeolite measured by the nitrogen adsorption method being is 30 or more at a relative pressure of 0.2 to 0.7.

2. The catalyst for producing a light olefin according to claim 1, wherein the pentasil-type zeolite has an absorption maximum between 3650 cm⁻¹ and 3710 cm⁻¹ in an infrared absorption spectroscopic measurement by Fourier transform infrared spectroscopy.

3. The catalyst for producing a light olefin according to claim 1, wherein the pentasil-type zeolite has an MFI structure.

4. The catalyst for producing a light olefin according to claim 1, wherein the silicon atoms and aluminum atoms contained in the pentasil-type zeolite satisfy the following atomic ratio:
[silicon atom/aluminum atom]=20 to 300.

5. The catalyst for producing a light olefin according to claim 1, wherein the catalyst is obtained by a hydrothermal synthesis at a temperature of 150° C. or lower.

6. The catalyst for producing a light olefin according to claim 1, wherein the catalyst is obtained by a hydrothermal synthesis using an organic silicon compound.

7. A method for producing a light olefin which uses the catalyst for producing a light olefin according to claim 1.

8. The method for producing a light olefin according to claim 7, wherein a light olefin is produced by reacting an oxygen-containing organic compound having 1 to 4 carbon atoms and the catalyst for producing a light olefin.

9. The method for producing a light olefin according to claim 8, wherein the oxygen-containing organic compound having 1 to 4 carbon atoms comprises at least one of methanol, dimethyl ether and ethanol.

10. The method for producing a light olefin according to claim 8, wherein steam is supplied to the oxygen-containing organic compound so that the weight ratio of the steam to the oxygen-containing organic compound satisfies the following equation:
[steam/oxygen-containing organic compound]=0.1 to 10.
